(19) United States
(12) Patent Application Publication
(10) Pub. No.: US 2006/0257463 A1
(43) Pub. Date: Nov. 16, 2006

(54) Transmucosal delivery of cannabinoids

(75) Inventors: Mahmoud Elsohly, Oxford, MS (US); Michael A. Repka, Oxford, MS (US)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(73) Assignee: UNIVERSITY OF MISSISSIPPI, University, MS (US)

(21) Appl. No.: 10/516,188

(22) PCT Filed: May 30, 2003
(86) PCT No.: PCT/US03/16812

§ 371 Date: Aug. 17, 2005

Related U.S. Application Data
(60) Provisional application No. 60/384,735, filed on May 31, 2002.

(51) Int. Cl. A61K 36/88; A61K 31/353; A61K 9/70

(52) U.S. Cl. 424/449; 424/774; 514/454

(57) ABSTRACT

A method of transmucosally delivering a cannabinoid to a subject in need of such treatment comprising the steps of: administering to the subject a transmucosal preparation containing the cannabinoid wherein said transmucosal preparation is made by incorporating an effective amount of the cannabinoid via hot-melt extrusion technology, hot-melt molding, admixing or a solvent cast technique into a film matrix or a reservoir containing the cannabinoid, and attaching said transmucosal preparation to the mucosa of the subject.

### FIELD OF THE INVENTION

This invention pertains to methods and products for the transmucosal administration of cannabinoids. In addition, this invention concerns a system for delivering effective dosages of cannabinoids to one's bloodstream.

### BACKGROUND OF THE INVENTION

Products and methods for transdermally administering particular chemicals are known in the art. Several U.S. patents have issued for the transdermal application of chemicals, most recently for cannabis (Brooke, et al., U.S. Pat. No. 6,113,940). Other methods and products for transmucosally delivering chemicals are also known in the art. However, this invention expands the concept of transdermal delivery of cannabis and the transmucosal delivery of other chemicals. The physico-chemical properties of cannabinoids (low water solubility, questionable stability, poor bioavailability) have limited its oral bioavailability and bioavailability via other routes of absorption. Discoveries disclosed within this invention have enabled the production of stable transmucosal preparations, with increased bioadhesivity and the efficient delivery of the cannabinoids to a subject in need of such treatment. For example, hot-melt extrusion, hot-melt molding, admixing, solvent cast and other techniques lend themselves to transmucosal preparations and absorption applications are described herein. It has been discovered that Tetrahydrocannabinol (THC) and other lipophilic derivatives of cannabinoids have appreciable solubility in polyethylene glycol 400 (PEG 400, >20%). Other glycols (i.e. propylene glycol, glycerin) and other solvents amenable to hot-melt extrusion and/or solvent casting or other preparation techniques may also be utilized. In any case, THC, THC pro-drugs or THC metabolites or derivatives or analogs thereof may be solubilized in an appropriate solvent and incorporated into the transmucosal preparation. This discovery, in addition to the application of hot-melt extrusion technology, hot-melt molding, admixing and solvent cast techniques has aided the delivery of cannabinoids via the transmucosal route.

In addition, it has been thought that stability of cannabinoids for inclusion into transmucosal preparations was prohibitive. It has been reported that THC is very unstable at room temperature [1] and its primary degradant is cannabinol (CBN). It has also been reported that THC instability is accelerated by ultraviolet light and heat [1]. However, in'studies outlined in this patent application, over 98% of THC was recovered after processing the drug into THC transmucosal matrix patch systems [2]. These findings will be detailed later in this document.

Another discovery has been the increased bioadhesivity of a transmucosal preparation (film/matrix or reservoir) when cannabinoids are added to said preparation. Transmucosal matrix film/patch preparations (8% and 16% THC) attained a peak bioadhesive force greater than the same patch preparations without the cannabinoid. Also, the peak force increased statistically (p<0.05) with an increase in percentage THC (THC 8% vs. THC 16%).

In addition, it has been demonstrated that the release of THC to a subject may be controlled via use of the parent compound (sustained release) or a THC pro-drug, the hemisuccinate, for a more immediate release.

Numerous medicinal uses have been reported for the active ingredients of cannabinoids, including tetrahydrocannabinol (THC), cannabinol, cannabidiol and other cannabinoids. For the purpose of this patent application, “cannabinoid” is meant to include Tetrahydrocannabinol (THC), THC pro-drugs or THC metabolites or derivatives or analogs thereof. Medicinal uses of cannabis include (A) treatment of nausea associated with cancer and chemotherapy, (B) nausea, pain and other complications of AIDS, such as wasting syndrome; (C) glaucoma; (D) migraines; (E) rheumatic and osteo-arthritis; (F) muscle dysfunction associated with multiple sclerosis; (G) alcohol and other chemical dependence withdrawal symptoms; (E) extreme stress; (I) depression; (J) asthma; and (K) epileptic seizures [3-9]. Although there have been many suggested benefits of cannabis, those benefits could be explained based on the effects of Δ⁹-THC. To date, the most promising clinical applications approved by the Food and Drug Administration (FDA) are for the control of nausea and vomiting associated with chemotherapy and for appetite stimulation of AIDS patients suffering from anorexia and the associated wasting syndrome [3, 4]. THC, however, demonstrates other biological activities which lend themselves to possible additional therapeutic applications as outlined above. At the present time, only Marinol®, a synthetic form of tetrahydrocannabinol is available by prescription to patients.

In the pharmaceutical industry, hot-melt extrusion has been used in the production of different dosage forms and systems for just over a decade [10-15]. It has been demonstrated to be applicable to various dosage forms including granules, pellets, and tablets and has also provided numerous advantages in the production of films for both drug delivery and wound care applications. Hot-melt extrusion technologies offer numerous advantages over traditional methods. These include shorter and more efficient processing times to a final product, environmental advantages due to elimination of solvents in processing, and increased efficiency of drug delivery to the patient.

Thin films for transdermal/transmucosal (TD/IT) drug delivery devices and wound care applications are frequently produced via film casting utilizing organic or aqueous solvents. Aitken-Nichol, et al. [15] noted numerous disadvantages accompanying these techniques including long processing times and high costs. Gutierrez-Rocca, et al. in the study of cast films [16] demonstrated that the attainment of stable mechanical properties might be as long as two months, which ultimately affects the rate of release of drugs incorporated into the films. However, this invention demonstrates that a stable film with good cannabinoid bioavailability can be attained for transmucosal delivery via a solvent cast technique.

U.S. Pat. No. RE 33,093 to Schiraldi et al. describes a bioadhesive hot-melt extruded film for intra-oral drug delivery and the processing thereof. The film of Schiraldi et al. comprises essentially a bioadhesive layer consisting of 40-95% by weight of a hydroxypropylcellulose (HPC) having a molecular weight above 100,000, 5-60% of a homopolymer of ethylene oxide (PEO) 3,000,000 to 5,000,000, 0-10% of a water-insoluble polymer, a medicament and 2-10% of a plasticizer. However, no other bioadhesives are included in this patent (i.e. acrylic acid derivatives). In addition, the films could not be processed at molecular weights for HPC below 100,000 and for PEO, below 3,000,000. The film was made by a hot-melt extrusion process. Mooney, et al. (U.S. Pat. No. 6,072,100) also describe a medicament delivery system consisting of HPC, PEO, a water-soluble polymer derived from acrylic acid, a medicament and a plasticizer. However, in this system, the compositions were intended for topical or transdermal delivery only. Gurtler, et al. (U.S. Pat. No. 5,773,021) teaches the development of a bioadhesive ophthalmic insert. However, the ophthalmic insert requires the presence of a water-insoluble polymer.

In summary, no prior art addresses the delivery of THC via the transmucosal route, most likely due to the cannabinoids' physicochemical properties including its low bioavailability and once thought heat sensitivity. In addition, increased bioadhesivity of a transmucosal film/patch matrix or reservoir preparation was not anticipated to increase the system's residence time and thus ultimately increase bioavailability. It is an object of this invention to provide a transmucosal delivery system to administer cannabinoids, particularly, THC, THC pro-drugs or THC metabolites or derivatives or analogs thereof.

### SUMMARY OF THE INVENTION

Although a transdermal route of administration has been disclosed in a U.S. patent as indicated above, an endeavor of the present invention is to extend the medicinal use of cannabinoids through the use of an effective transmucosal route of administration. Particularly, hot-melt extrusion, hot-melt molding, admixing and solvent casting of a transmucosal device are of interest.

Although the chemical and physical properties of the cannabinoids have limited their bioavailability, it has been discovered that their bioavailability can be very effective via the described transmucosal delivery system in this invention. Polyethylene glycol 400 has been reported in the scientific literature to act as an absorption enhancer of the skin. It (and other solubilizers) may also serve as an absorption enhancer of cannabinoids for the mucosa, in addition to functioning as a solubilizer for the cannabinoid for the hot-melt extrusion, hot-melt molding and admixing processes or solvent cast techniques. The primary active ingredient of cannabis is THC, which is effective at relatively low doses. Due to its high lipophilicity, THC exhibits a strong tendency to bind to tissue and protein—thus making the discussed transmucosal applications plausible routes of delivery. Furthermore, mHC is rapidly metabolized in the body, such that concentration levels of the chemical in the bloodstream decreases rapidly if administered through inhalation methods. A transmucosal application, in contrast to inhalation methods allows for smaller dosages of THC to be administered over an extended period of time, thereby allowing the concentration levels of the drug in the blood stream to remain relatively constant. In addition, and of utmost importance, the smaller doses of the transmucosal route reduce the potential for abuse.

The present invention comprises a transmucosal device, such as, but not limited to, an intra-oral, labial or buccal patch, strip, covering, or related assembly of materials to deliver THC or other cannabinoids in a predetermined period of time. One purpose of the structure or method is to allow for controlled delivery of the active chemicals, such that plasma levels of the chemicals may be controlled in a safe, convenient and effective manner for the patient.

This invention also comprises the method of treating a patient with a transmucosal cannabinoid-containing preparation. Most conveniently, this is accomplished by application of the transmucosal structure described herein. Additional steps for increasing the permeability of the patient's mucosa may further comprise the method for transmucosally applying cannabinoids, such as the permeability enhancement of PEG 400 and/or other enhancers in which cannabinoids may be solubilized. Solubilizers (which may inherently be penetration or absorption enhancers) useful in the present invention include, for example, Polyethylene glycol (PEG), Propylene glycol, Dibutyl subacetate, Glycerol, Diethyl phthalate (phthalate esters), Triacetin, Citrate esters-triethyl citrate (TEC), Acetyltriethyl citrate (ATEC), tributyl citrate (TBC), acetyltributyl citrate (ATBC), Benzyl benzoate, Sorbitol, Xylitol, Miglyol (Glycerides), bis(2-ethyllhexyl) adipate, Mineral Oil, polyhydric alcohols such as glycerin and sorbitol, glycerol esters such as glycerol, triacetate; fatty acid triglycerides such as NEOBEE* M-5 and mineral oil, vegetable oils such as castor oil, etc., polyoxyethylene sorbitan, fatty acid esters such as TWEENS, polyoxyethylene monoalkyl ethers such as BRIJ and MYRJ series, sucrose monoesters, lanolin esters, lanolin ethers. Also included as solubilizers for the cannabinoids are organic solvents, such as ethanol, benzene and the like, which may be utilized in solvent cast techniques.

### DETAILED DESCRIPTION OF THE INVENTION

The invention includes a transmucosal preparation wherein said transmucosal preparation is made by incorporating an effective amount of a cannabinoid by solubilizing or dispersing the cannabinoid into the transmucosal cannabinoid-containing preparation. The transmucosal preparation may be produced via hot-melt extrusion, hot-melt molding, admixing or utilizing a solvent cast technique. The invention may include a matrix patch or reservoir means for retaining and dispersing the active ingredient(s). The matrix may include, but not be limited to polytheylene oxide (PolyOx®), polyvinylpyrrolidone (Kolidon®), hydroxypropyl cellulose (Klucel®), ethyl cellulose, methylcellulose, alkylcelluloses, veegums clays, alginates, PVP, alginic acid, carboxymethylcellulose calcium, microcrystalline cellulose (e.g., Avicel™), polacrillin potassium (e.g., Amberlite™), sodium alginate, corn starch, potato starch, pregelatnized starch, modified starch, cellulosic agents, montmorrilonite clays (e.g., bentonite), gums, agar, locust bean gum, gum karaya, pecitin, tragacanth, and other matrix formers known to those skilled in the art. In one embodiment of the invention, the hot-melt extruded matrix film contains a solid dispersion or solution of the active cannabinoid. This matrix may optionally contain a bioadhesive (such as a carbopol, polycarbophil, chitosan or others known to those skilled in the art—to further enhance the bioadhesivity of the cannabinoid itself) or a bioadhesive layer may be laminated onto the matrix film or patch containing the cannabinoid. In addition, an impermeable backing layer may be incorporated to insure unidirectional flow of the drug through the patient's mucosa. In some cases a rate controlling film or membrane may also be laminated or sprayed onto the cannabinoid-containing matrix to further control the rate of release of the actives.

The transmucosal preparation will preferably contain a ‘penetration enhancer’ (which may also be referred to as an absorption enhancer or permeability enhancer). These penetration enhancers may include bile salts, such as sodium deoxycholate, sodium glycodeoxycholate, sodium taurocholate and sodium glycocholate, surfactants such as sodium lauryl sulfate, polysorbate 80, laureth-9, benzalkonium chloride, cetylpyridinium chloride and polyoxyethylene monoalkyl ethers such as the BRIJ® and MYRJ® series. Additional penetration enhancers for inclusion in the embodiment include benzoic acids, such as sodium salicylate and methoxy salicylate, fatty acids, such as lauric acid, oleic acid, undecanoic acid and methyl oleate, fatty alcohols, such as octanol and nonanol, laurocapram, the polyols, propylene glycol and glycerin, cyclodextrins, the sulfoxides, such as dimethyl sulfoxide and dodecyl methyl sulfoxide, the terpenes, such as menthol, thymol and limonene, urea, chitosan and other natural and synthetic polymers.

In yet another embodiment of the invention, a reservoir containing the cannabinoid and other rate controlling measures overlying an extruded matrix layer or layers, covered by an impermeable backing layer is disclosed. The rate controlling means particularly regulates flux, in addition to the matrix layer or layers, of the cannabinoid to the mucosa. In this embodiment, the cannabinoid is dissolved in an appropriate solvent or polymer containing solution or suspension that will then be control released as the extruded matrix layer hydrates and erodes so that mucosal absorption is attained.

The rate controlling means may comprise a nonporous or porous polymer membrane for controlling the diffusion rate of cannabinoids. The reservoir means may also comprise a polymer matrix material, hot-melt extruded or otherwise that suspends the cannabinoid and releases it in a controlled manner. The flux of the polymer matrix material may further be regulated by the rate controlling membrane.

The present invention provides a bioadhesive system that is an effective, feasible, and convenient intra-oral drug delivery system for applying and delivering controlled dosages of canmabinoid agents through or into the oral cavity. This invention may also be extended to controlled drug delivery in gynecological (vaginal), nasal, sinus, and ophthalmic applications. Preferred processes are hot-melt extrusion or hot-melt molding which generally provide shorter and more efficient processing times to a final product, environmental advantages due to elimination of solvents in processing, better stability and increased efficiency of drug delivery to the patient. However, an admixed system and a solvent cast system may be employed.

This invention is generally directed to an extruded single or multi-layered laminated film matrix containing the cannabinoid that can be cut or formed into almost unlimited shapes and sizes, depending on the application and dosage intended. Matrices of different thickness and shapes may be prepared by changing the extrusion die, varying the extrusion rate or varying the film tension between the chill-roll or take-off roll and the extruder.

The transmucosal device film or films (in the case of co-extrusion or layering) generally comprises at least one water-soluble, water-swellable or water-insoluble thermoplastic polymer such as, but not limited to, hydroxypropylcellulose, polyethylene oxide, homopolymers and copolymers of carboxymethyl cellulose, ethylcellulose, hydroxyethyl cellulose and hydroxymethyl cellulose with a cannabinoid or multiple cannabinoids as the medicament(s). The hot-melt extruded or hot-melt molded matrix may also comprise as bioadhesives such as water-soluble or water-swellable polymers derived from acrylic acid or a pharmaceutically acceptable salt thereof, such as the polyacrylic acid polymers, including carbomers, polycarbophils and/or water-soluble salts of a co-polymer of methyl vinyl ether and maleic acid or anhydride (Gantrez MS-955). The film can also comprise one or more pH adjusting agents, additives (such as penetration enhancers), and/or hydrophobic polymers that may render the film useful for particular transmucosal applications. The film is generally used for controlled delivery of cannabinoids to the patient.

The film formulations of the invention will adhere to mucosal surfaces (oral, vaginal, etc.) when wet. This bioadhesion is enhanced by the discovered adhesive properties of the cannabinoids when incorporated into a transmucosal preparation, via hot-melt extrusion, hot-melt molding, admixing or solvent cast techniques.

The invention includes a transmucosal preparation wherein said transmucosal preparation is made by incorporating an effective amount of a cannabinoid by solubilizing or dispersing the cannabinoid into the cannabinoid preparation. The preparation may be produced via hot-melt extrusion, hot-melt molding, admixing or utilizing a solvent cast technique. The preparation of this invention which is useful for delivering cannabinoids through the mucosal tissue may also comprise, other than stated above, additives which may make the matrix more flexible or thermoplastic.

The transmucosal preparation can also comprise one or more pH-adjusting agents to improve stability and solubility. Also the pH modifying agents can control cannabinoid release and enhance bioadhesion. A pH-adjusting agent can include, by way of example and without limitation, an organic acid or base, an alpha-hydroxy acid, or a beta-hydroxy acid. Suitable agents include tartaric acid, citric acid, fumaric acid, succinic acid and others known to those of ordinary skill in the art.

The transmucosal preparation can also comprise one or more cross-linking agents to reduce matrix erosion time, control release of the cannabinoid or enhance bioadhesion. A cross-linking agent can include, by way of example and without limitation, an organic acid, an alpha-hydroxy acid, or a beta-hemolytic-hydroxy acid. Suitable cross-linking agents include tartaric acid, citric acid, fumaric acid, succinic acid and others known to those of ordinary skill in the art.

The transmucosal preparation may also contain other components that modify the extrusion, molding or casting characteristics or physical properties of the matrix. Such other components are well known to those of ordinary skill in the pharmaceutical sciences and include, for example, polyethylene, xylitol, sucrose, surface-active agents, others mown to those skilled in the art, and combinations thereof.

The transmucosal preparation of the present invention can also include super-disintegrants or absorbents. Examples of such are sodium starch glycolate (Explotab™, Primojel™) and croscarmellose sodium (Ac-Di-Sol®). Other suitable absorbents include cross-linked PVP (Polyplasdone™ XL 10), clays, alginates, corn starch, potato starch, pregelatinized starch, modified starch, cellulosic agents, montmorrilonite clays (bentonite), gums, agar, locust bean gum, gum karaya, pectin, tragacanth, and other disintegrants known to those of ordinary skill in the art.

The transmucosal preparation can also include one or more of each of a pH buffering agent, an antioxidant, chelating agent, stabilizer, surfactant, preservative, paraben, flavor, colorant, fragrance and combinations thereof.

pH buffering agents include alkalinizing agents, acidifying agents and salts thereof. A buffering agent is used to resist change in pH upon dilution or addition of acid or alkali. Such compounds include, by way of example and without limitation, potassium metaphosphate, potassium phosphate, monobasic sodium acetate and sodium citrate anhydrous and dihydrate, salts of inorganic or organic acids, salts of inorganic or organic bases, and others known to those of ordinary skill in the art.

As used herein, the term “acidifying agent” is intended to mean a compound used to provide acidic medium for product stability. Such compounds include, by way of example and without limitation, acetic acid, amino acids, citric acid, fumaric acid and alpha hydroxy acids, such as ascorbic acid, and inorganic acids such as hydrochloric acid and nitric acid and others known to those of ordinary skill in the art.

As used herein, the term “alkalinizing agent” is intended to mean a compound used to provide alkaline medium for product stability. Such compounds include, by way of example and without limitation, ammonia solution, ammonium carbonate, diethanolamine, monethanolamine, potassium hydroxide, sodium borate, sodium carbonate, sodium bicarbonate, sodium hydroxide, triethanolamine, and trolamine and others known to those of ordinary skill in the art.

The transmucosal preparation of the invention can include a chelating agent. Suitable chelating agents include EDTA, polycarboxylic acids, polyamines, derivatives thereof, and others known to those of ordinary skill in the art.

The transmucosal preparation of the invention can include a surfactant. Suitable surfactants include sucrose stearate, Vitamin E derivatives, sodium lauryl sulfate, dioctyl sodium sulfosuccinate, and others known to those of ordinary skill in the art.

The transmucosal preparation of the invention can include a preservative. Preservatives include compounds used to prevent the growth of microorganisms. Suitable preservatives include, by way of example and without limitation, benzalkonium chloride, propyl paraben, methyl paraben, benzyl alcohol, cetylpridinium chloride, chlorobutanol, sorbic acid, phenol, phenylethyl alcohol, phenylmercuric nitrate and thimerosal and others known to those of ordinary skill in the art.

As used herein, the term “flavorant”, “flavor” or “fragrance” is intended to mean a compound used to impart a pleasant flavor and often odor to a pharmaceutical preparation. In addition to the natural flavorants, many synthetic flavorants are also used. Such compounds include, by way of example and without limitation, anise oil, cinnamon oil, cocoa, menthol, orange oil, peppermint oil and vanillin and others known to those of ordinary skill in the art. Flavors incorporated in the composition may be chosen from synthetic flavor oils and flavoring aromatics and/or natural oils, extract from plants, leaves, flowers, fruits and so forth and combinations thereof. These may include oil of wintergreen, clove oil, bay oil, anise oil, eucalyptus, thyme oil cedar leaf oil, oil of nutmeg, oil of sage, oil of bitter almonds and cassia oil. Also useful as flavors are vanilla, citrus oils, including lemon, orange, lime and grapefruit, and fruit essences, including grape, apple, pear, peach, strawberry, raspberry, cherry, plum, apricot, and so forth. Flavors that have been found to be particularly useful include commercially available orange, grape, cherry, and bubble gum flavors and mixtures thereof. The amount of flavoring may depend on a number of factors, including the organoleptic effect desired.

As used herein, the term “colorant” is intended to mean a compound used to impart color to solid pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide red. Other suitable colorants include titanium dioxide and natural coloring agents such as grape extract, beet red powder, carmine, turmeric, paprika, and others known to those of ordinary skill in the art.

As used herein, the term “antioxidant” is intended to mean an agent that inhibits oxidation and thus is used to prevent the deterioration of preparations by oxidation. These compounds include, by way of example and without limitation, ascorbic acid, ascorbyl palnitate, butylated hydroxyanisole (BHA), butylated hydroxytoluene (B3HT), hypophophorous acid, monothioglycerol, sodium ascorbate, sodium formaldehyde sulfoxylate and sodium metabisulfate and others known to those of ordinary skill in the art. Other suitable antioxidants include, for example, vitamin C, sodium bisulfite, vitamin E and its derivatives, propyl gallate, a sulfite derivative, and others known to those of ordinary skill in the art.

Embodiments of the transmucosal preparation that provide a controlled release of an agent may contain a release rate modifier. Suitable release rate modifiers include hydroxypropylcellulose (BpC), poly(ethylene oxide) (PEO), hydroxypropyl methlcellulose (HPMC), ethylcellulose, cellulosic polymers, acrylic polymers, fat, waxes, lipid, or a combination thereof In some embodiments, the release rate modifier is polycarbophil, carbomer or a polysaccharide.

The ingredients and chemicals used for the production of the transmucosal preparation used in this invention are of acceptable quality, preferably pharmaceutically acceptable quality. The cannabinoid-containing transmucosal preparation is homogenous and pharmaceutically acceptable.

The foregoing will be better understood with reference to the following examples that detail certain procedures for the preparation of formulations according to the present invention. All references made to these examples are for the purposes of illustration. They are not to be considered limiting as to the scope and nature of the present invention.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Example #1 was prepared by hot-melt molding. The Tetrahydrocannabinol (THC) was dissolved within the Polytheylene glycol 400. The other Inner matrix components were then mixed and heated to approximately 140° C. and homogeneously blended. The solubilized THC was then slowly added to the heated admixture and dispersed. The resulting molten matrix was then poured into a film mold to obtain a uniformly thick (approximately 1.5 mm) film after slowly cooling. The backing layer was adhered with 40° C. heating. The Outer backing ingredients were heated (90° C.), mixed and molded separately.

### EXAMPLE #1

### Transmucosal Device for THC Delivery via the Oral Mucosa

| | Inner | Outer |
|---|---|---|
| Ingredients | matrix (%) | backing layer |
| Hydroxypropylcellulose | 75.90 | 37.00 |
| (Klucel ®-EF) | | |
| Polycarbophil (Noveon ® AA-1) | 5.00 | |
| Polyethylene glycol 400 | 8.00 | |
| Tartaric Acid | 3.00 | |
| BHT | 0.10 | |
| Tetrahydrocannabinol (THC) | 8.00 | |
| Ethyl cellulose | | 60.00 |
| Eudragit ® E-100 | | 2.00 |
| Polyethylene glycol 3350 | | 1.00 |

Example #1 above contains the solubilizer PEG 400, which may also function as a penetration enhancer. An additional example (Example #1a) may include the above formula with the bile salt penetration enhancer, sodium deoxycholate, at the 5% level.

### EXAMPLE #2, #3, #4

| Drug/Chemical (% w/w) | #2 | #3 | #4 |
|---|---|---|---|
| Hydroxypropyl cellulose (Avg MW: 80,000) | 15.5 | 11.5 | 3.5 |
| Polyethylene Oxide (Avg MW: 200,000) | 80.4 | 80.4 | 80.4 |
| Vitamin E succinate | 0.1 | 0.1 | 0.1 |
| Tetrahydrocannabinol Hemiglutarate | 4 | 8 | 16 |
| (THC Pro-drug) | | | |

Example #2-#4 were prepared via a solvent cast technique. The Tetrahydrocannabinol Hemiglutarrate (THC-HG) was dissolved in ethanol (10% w/w of THC-HG). The HPC and PEO were then admixed with the Vitamin E succinate via solvation. Then the THC-HG solution was slowly added to the polymeric dispersion. The resulting dispersion was added to a film forming mold and the solvent was evaporated off. The resulting transmucosal preparation was homogenously dispersed with the cannabinoid pro-drug.

Example #5 and #6 were prepared using hot-melt extrusion techniques. The formulas are listed below. The PEO, PVP and Vitamin E TPGS were dry blended in a V-blender. The THC and the THC-HS were solubilized in the PEG 400 and immediately sprayed into the dry blend with continuous mixing. The resulting blend was then hot-melt extruded into films. The highest extrusion temperature was 150° C. and residence time in the barrel was approximately 2 minutes. The resulting transmucosal preparations were approximately 1.0 mm in thickness and both contained over 98% of the original theoretical percent of drug within the formulation.

### EXAMPLES #5 & #6

| Drug/Chemical (% w/w) | #5 | #6 |
|---|---|---|
| Polyethylene Oxide (Avg MW: 1,000,000) | 68.0 | 68.0 |
| Polyvinylpyrrolidone (Kollidon) | 10.0 | 10.0 |
| Polyethylene glycol (PEG 400) | 11.0 | 11.0 |
| Vitamin E TPGS | 3.0 | 3.0 |
| Tetrahydrocannabinol (THC) | 8.0 | — |
| Tetrahydrocannabinol Hemisuccinate | — | 8.0 |
| (THC-HS) | | |

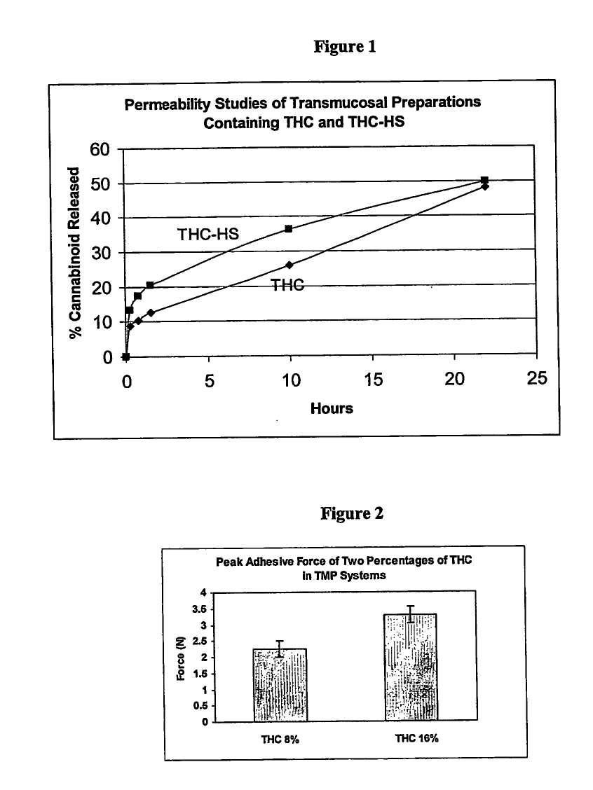

Diffusion studies of the transmucosal preparation films in Examples #5 and #6 were performed using a PermeGear, Model V9, 9 Cell System. Modified Franz cells were employed using thinly-excised rabbit mucosa as the diffusion membrane. Diffusion media was a diffusion buffer system of Brij® 3.0% (pH=7.2) which was determined by previous testing. FIG. 1 illustrates the results of these studies. As can be seen by the illustration, the THC-HS exhibited a more immediate release with controlled diffusion for over 22 hours. Example #5 (THC) demonstrated a slower release with approximately 50% theoretical drug released at 22 hours. Both formulations have clinical applications for different therapeutic objectives.

It has been reported that THC is very unstable at room temperature [1] and its primary degradant is cannabinol (CBN). It has also been reported that THC instability is accelerated by ultraviolet light and heat [1]. However, the studies within this invention have demonstrated that over 98% of THC was recovered after processing the drug into mHC Transmucosal Matrix Patch (IMP) Systems [2].

Hot-melt molding of four batches of the following formulations containing THC was performed. The TWP systems were obtained with a thickness range from 0.3 mm to 3.0 mm. Melt temperature for the formulas ranged from 90° C. to 140° C. Table I outlines the formulas used for stability study testing.

TABLE I
Formulations of extruded TMP systems

| | | | TMP- | TMP- |
|---|---|---|---|---|
| Drag/Chemical (% w/w) | TMP-8 | TMP-16 | 8-DC | 16-DC |
| Hydroxypropyl cellulose | 20.0 | 10.0 | 20.0 | 10.0 |
| (Avg MW: 80,000) | | | | |
| Hydroxypropyl cellulose | 41.23 | 51.23 | 43.23 | 45.23 |
| (Avg MW: 140,000) | | | | |
| Polyethylene Glycol | 12.0 | 12.0 | 10.0 | 8.0 |
| 400, NF | | | | |
| Polyethylene Oxide | 13.0 | 10.0 | 13.0 | 10.0 |
| (Avg MW: 200,000) | | | | |
| Propylparaben NF | 0.02 | 0.02 | 0.02 | 0.02 |
| Methylparaben NF | 0.20 | 0.20 | 0.20 | 0.20 |
| Butylated Hydroxy- | 0.05 | 0.05 | 0.05 | 0.05 |
| toluene NF | | | | |
| Carbomer (Carbopol | 5.00 | 5.00 | 5.00 | 5.00 |
| 971P) | | | | |
| Citric Acid | 0.5 | 0.5 | 0.5 | 0.5 |
| Sodium Deoxycholate | — | — | 5.0 | 5.0 |
| Δ⁹-Tetrahydro- | 8.0 | 16.0 | 8.0 | 16.0 |
| cannabinol | | | | |

Table II illustrates the percent drug remaining (via HPLC) in the four formulations within 24 hours post-extrusion and after 12 months. This preliminary data is encouraging in that it indicates that all four formulations have greater than 96% theoretical drug remaining after 12 months. It has recently been demonstrated that significant THC degradation does not occur until the cannabinoid is hot-melt processed at 200° C. for 20 minutes [2]. In this study, when Tetrahydrocannabinol was incorporated into cellulosic (Kucel®) matrix films processed at 120, 160 and 200° C. (for 20 minutes), THC degradation within the hot-mold matrix was found to be 1.8, 2.3 and 4.3%, respectively. This is a significant finding in that during hot-melt extrusion, hot-melt molding and admixing processes involving heat, the cannabinoid is only subjected to temperatures from 90-140° C. for 2 to 7 minutes. In summary, with proper processing, packaging and storage conditions, THC and other cannabinoids are good candidates for transmucosal delivery preparations involving the judicious application of heat.

TABLE II
Percent drug remaining in the TMP systems
post-extrusion (25° C., 60% RH)

| | % Theoretical Drug | % Theoretical Drug |
|---|---|---|
| Formulations | Remaining (t = 0) | Remaining (t = 12 months) |
| TMP-8 | 98.2 ± 1.7 | 96.3 ± 2.1 |
| TMP-16 | 99.1 ± 2.2 | 97.9 ± 1.2 |
| TMP-8-DC | 98.8 ± 1.3 | 96.2 ± 1.8 |
| TMP-16-DC | 99.2 ± 2.7 | 96.2 ± 1.9 |

Bioadhesive experiments were conducted on the THC Pro-drug formulation systems using a TA.XT2i Texture Analyzer equipped with Texture Expert™ software to produce force-deflection profiles. The substrate used for bioadhesion testing was rabbit intestinal mucosa All formulations were prepared by an ethanol solvent cast method. TMP 8% & TMP 16% attained a peak force of 2.5 N and 3.4 N, respectively. The peak bioadhesive force of both THC incorporated systems increased statistically (p<0.05) with an increase in percentage THC compared to the control (0% TMP, 1.9N). These results indicate that the % of mHC incorporated in the systems have relevance for clinical studies in that incorporation of the cannabinoid increases the residence time of the transmucosal preparation and thus increases bioavailability. FIG. 2 illustrates these bioadhesion results. Table III represents the formulations for the transmucosal matrices.

TABLE III
Formulations of TMP systems via ethanol solvent casting

| Drug/Chemical (% w/w) | 0% TMP | 8% TMP | 16% TMP |
|---|---|---|---|
| Hydroxypropyl cellulose | 19.5 | 11.5 | 3.5 |
| (Avg MW: 140,000) | | | |
| Polyethylene Oxide | 80.4 | 80.4 | 80.4 |
| (Avg MW: 200,000) | | | |
| Butylated | 0.1 | 0.1 | 0.1 |
| Hydroxytoluene NF | | | |
| Tetrahydrocannabinol | 0 | 8 | 16 |
| Hemisuccinate | | | |
| (THC Pro-drug) | | | |

It will be understood by those skilled in the art that various modifications and substitutions may be made to the invention as described above without departing from the spirit and scope of the invention. Accordingly, it is understood that the present invention has been described by way of illustration and not limitation.

1. Ross, S. A. and M. A. ElSolhy, CBN and delta-9-THC concentration ratio as an indicator of the age of stored marijuana samples. Bulletin on Narcotics, 1997/1998. 49/50(1&2): p. 139-147.

2. Munjal, M. and M. A. Repka Effect of processing temperature on the stability of delta-9-Tetrahydrocannabinol in Klucel polymer matrix systems in Annual Meeting of the American Association of Pharmaceutical Scientists. 2002. Toronto, Canada

3. Marijuana and Medicine: Assessing the Science Base, ed. J. E. Joy, S. J. Watson, and J. A. Benson. 1999, Washington, D.C.: National Academy Press.

4. Martin, B. R., The use of cannabinoids in patients with chronic illness. U.S. Pharmacist, 2002. 1: p. 61-72.

5. ElSolhy, M. A., E. Harland, and C. W. Waller, Cannabinoids in glaucoma II: The effect of different cannabinoids on the intraocular pressure of the rabbit. Curr Eye Res, 1984. 3(6): p. 841-850.

6. El-Mallakh, R. S., Marihuana and migraine. Headache, 1987. 2(8): p. 442-443.

7. Maurer, M., et al., Delta-9-tetrahydrocannabinol shows antispastic and analgesic effects in a single case double-blind trial Eur. Arch. Psychiatry Clin. Neurosci., 1990. 240(1): p. 1-4.

8. McLendon, D. M., R. T. Harris, and W. F. Maule, Suppression of the cardiac conditioned response by delta-9-tetrahydrocannabinol: A comparison with other drugs. Psychopharmacology, 1976. 50(2): p. 159-163.

9. Mattes, R. D., et al., Bypassing the first-pass effect for the therapeutic use of cannabinoids. Pharmacol., Biochem., Behav., 1994. 49(1): p. 187-195.

10. Follonier, N., B. Doelker, and E. T. Cole, Evaluation of hot-melt extrusion as a new technique for the production of polymer-based pellets for sustained release capsules containing high loadings of freely soluble drugs. Drug Dev Ind Pharm, 1994. 20: p. 1323-1339.

11. Repka, M. A., et al., Influence of plasticizers and drugs on the physical-mechanical properties of hydroxypropylcellulose films prepared by hot-melt extrusion. Drug Dev Ind Pharm, 1999. 25: p. 625-633.

12. Repka, M. A. and J. W. McGinity, Bioadhesive Properties of Hydroxypropylcellulose Topical Films Produced by Hot-Melt Extrusion. J Control Release, 2001. 70: p. 341-351.

13. Repka, M. A. and J. W. McGinity, Physical-mechanical, moisture absorption and bioadhesive properties of hydroxypropylcellulose hot-melt extruded films. Biomaterials, 2000. 21(14): p. 1509-1517.

14. Follonier, N., E. Doelker, and E. T. Cole, Various ways of modulating the release of diltiazem hydrochloride from hot-melt extruded sustained release pellets prepared using polymeric materials. J Controlled Release, 1995. 36: p. 243-250.

15. Aitken-Nichol, C., F. Zhang, and J. W. McGinity, Hot melt extrusion of acrylic films. Pharm Res, 1996. 13: p. 804-808.

16. Gutierrez-Rocca, J. C. and J. W. McGinity, Influence of aging on the physical-mechanical properties of acrylic resin films cast from aqueous dispersions and organic solutions. Drug Dev Ind Pharm, 1993. 19: p. 315-332.

What is claimed:

1. A method of transmucosally delivering a cannabinoid to a subject in need of such treatment comprising the steps of: administering to the subject a transmucosal preparation containing the cannabinoid wherein said transmucosal preparation is made by incorporating an effective amount of the cannabinoid via hot-melt extrusion technology, hot-melt molding, admixing or a solvent cast technique into a film matrix or a reservoir containing the cannabinoid, and attaching said transmucosal preparation to the mucosa of the subject.

2. The method of claim 1 wherein the transmucosal preparation comprises a solubilizer for the cannabinoid and the cannabinoid comprises THC, THC pro-drugs or THC metabolites or derivatives or analogs thereof.

3. The method of claim 2, wherein the transmucosal preparation further contains an absorption or penetration enhancer.

4. The method of claim 1, wherein the preparation optionally comprising a backing layer.

5. The method of claim 4, wherein the backing layer is a patch, strip, bandage or covering (laminated or sprayed) for allowing unidirectional mucosal flow of the cannabinoid.

6. The method of claim 1, comprising attaching the transmucosal preparation to buccal or labial mucosa (or other mucosal area) of said subject so that the cannabinoid can be absorbed systemically.

7. An article useful for transmucosal delivery of a cannabinoid to a subject comprising a transmucosal preparation having a backing layer and a reservoir means said reservoir means containing the cannabinoid.

8. An article according to claim 7 where the cannabinoid comprises THC, THC pro-drugs or THC metabolites or derivatives or analogs thereof.

9. The article of claim 7 wherein the reservoir means is any one or combination of a member of the group consisting of a cavity, matrix material and film.

10. An article for administering a cannabinoid to a subject's mucosa, comprising: at least one layer of a matrix material suitable for attachment to the mucosa; and, a cannabinoid in said matrix material, said preparation being capable of delivering an effective amount of the cannabinoid through the mucosa.

11. The article of claim 10 wherein the matrix material comprises a backing material including a reservoir means for retaining said cannabinoid.

12. The article of claim 11 wherein said reservoir means comprises a polymer matrix attached to said material, said cannabinoid being dissolved or suspended in said polymer matrix.

13. The article of claim 12 wherein said reservoir means comprises a cavity formed in said backing material, said cannabinoid being contained in said cavity.

14. The article of claim 13 wherein a rate controlling means overlies said cavity for regulating the flow of the cannabinoid to said mucosa.

15. The article of claim 14 wherein said rate controlling means comprises a member selected from the group consisting of a porous or non-porous membrane, a polymer film, or a polymer membrane.

16. The article of claim 10 wherein said cannabinoid comprises a liquid or gel carrier combined with the cannabinoid.

17. The article of claim 10 wherein said matrix material includes adhesive means for attaching said structure to the mucosa.

18. The method of claim 1 wherein said transmucosal preparation includes an adhesive which is adapted to adhere said transmucosal preparation to the mucosa of the subject.

19. The method of claim 6 further comprising, maintaining said transmucosal preparation in contact with the mucosa for an appropriate period of time to control the delivery of the cannabinoid(s).

20. A method of delivering a cannabinoid to a subject in need of such treatment comprising preparing a transmucosal preparation containing the cannabinoid wherein the transmucosal preparation is adapted for administration of the cannabinoid through the mucosa of the subject by attaching the transmucosal preparation to the subject's mucosa.
